(19) United States
(12) Patent Application Publication
(10) Pub. No.: US 2007/0270641 A1
(43) Pub. Date: Nov. 22, 2007

(54) Capsule Endoscope System and Capsule Endoscope

(76) Inventors: Seiichiro Kimoto, Tokyo (JP); Hatsuo Shimizu, Tokyo (JP)

Correspondence Address: SCULLY SCOTT MURPHY & PRESSER, PC, 400 GARDEN CITY PLAZA, SUITE 300, GARDEN CITY, NY, 11530, US

(21) Appl. No.: 11/665,289

(22) PCT Filed: Oct. 15, 2004
(86) PCT No.: PCT/JP04/15280

§ 371 Date: Apr. 13, 2007

(51) Int. Cl. A61B 1/04

(52) U.S. Cl. 600/109

(57) ABSTRACT

In a capsule endoscope system (1) that includes a capsule endoscope (10) including a transmitting unit (141, 142) for transmitting captured image data and a display unit (5a) for visualizing and displaying the image, and including an external device (4), units (131 to 133) are included for initiating power supply to a capsule main body (14) in such a state that an imaging window (120b) and an illumination window (120a) are covered by an internal package (70), an image-data-correction value is acquired from image data obtained by performing a first imaging of a chart (71) for acquiring an image-data correction value, the chart (71) arranged inside the package so that the chart faces the imaging window (120b), with the internal package (70) covered, image data obtained by performing a second imaging is corrected by the correction value, and the image data is visualized to be displayed on the display unit (5a).

### TECHNICAL FIELD

The present invention relates to a swallowable medical endoscope system (hereinafter, “capsule endoscope system”).

### BACKGROUND ART

Recently, such a capsule endoscope system has been proposed that enables to observe a body cavity image by using a capsule endoscope that includes in a capsule-shaped case, an imaging unit including a solid-state image sensor such as a charge-coupled device (CCD), and an illuminating unit including a light emitter such as a light emitting diode (LED).

More specifically, when a subject swallows the capsule endoscope from a mouth, an image of the body cavity such as a stomach and an intestine is captured by the capsule endoscope while the capsule endoscope travels through inside the body cavity. An image data is transmitted to an external device outside the body cavity and visualized by using, for example, a monitor, to realize an observation of the body cavity image.

The image data acquired by the capsule endoscope is generated by performing a signal process such as a data correction (i.e., a color correction and a y correction) in the capsule endoscope or in a receiving device.

An image-data correction value used for the image-data correction is set in advance in accordance with a type of a light source that structures the illuminating unit or with a position of an imaging lens in the imaging unit.

### DISCLOSURE OF INVENTION

### Problem to be Solved by the Invention

However, because a color of the light source is slightly different and a position of the imaging lens varies, with respect to each product (capsule endoscope), there still is a possibility that an image visualized by performing a correction using the predetermined correction value does not have an optimal color reproduction and an optimal resolution in every capsule endoscope.

The image-data correction value can be acquired by capturing an image of a chart for acquiring the image-data correction value, before the capsule endoscope is introduced into the body cavity. However, when the above image is captured, an illumination in a room is likely to be input, and the image-data correction value obtained in the above situation is not suitable for capturing an image inside the body cavity, that is, not suitable for capturing an image with a light source of a light emitter in the capsule endoscope. Further, it is difficult to capture an image of the chart with the light source of the light emitter in the capsule endoscope.

An object of the present invention is to solve the above problems and to provide a capsule endoscope system that enables to easily acquire the most optimal image-data correction value with respect to each product (capsule endoscope) so that the visualized image has the optimal color reproduction and resolution, and further, to make it possible to verify whether the image-data correction value is most optimal.

### Means for Solving Problem

Namely, a capsule endoscope system includes a capsule endoscope that contains an imaging unit, an illuminating unit capable of illuminating an imaging target region, and a transmitting unit capable of transmitting image data obtained by the imaging unit to an outside; and an external device separated from the capsule endoscope, the external device including a receiving unit that receives the image data and a display unit capable of visualizing and displaying the image captured by the imaging unit; a package that covers at least one of an imaging window and an illumination window of the capsule endoscope, in such a state that an external light can be shielded; a power-supply initiating unit capable of initiating a power supply to the imaging unit and the illuminating unit, in such a state that the imaging window and the illumination window are covered by the package; and a chart portion for acquiring an image-data correction value, arranged inside the package so that the chart portion faces the imaging window, wherein the image-data correction value is acquired from an image data obtained by performing a first imaging of the chart portion, in such a state that the imaging window and the illumination window are covered by the package, an image data obtained by performing a second imaging after the first imaging is corrected by the obtained image-data correction value, in such a state that the imaging window and the illumination window are covered by the package, and the image data is visualized and displayed on the display unit.

At this state, the image-data correction value is acquired in each of the capsule endoscopes, before the capsule endoscope is introduced into a body cavity, in such a state that the imaging window and the illumination window are covered by the package. Accordingly, the image-data correction value becomes optimal. Further, the optimal image-data correction value can easily be acquired and power supply can be initiated to the capsule endoscope at a substantially same timing. Moreover, it becomes possible to verify whether the correction value is optimal, by visualizing a secondly captured image by the display unit.

### EFFECT OF THE INVENTION

With a capsule endoscope according to an embodiment of the present invention, an image-data correction value is acquired in the capsule endoscope with respect to each product before the capsule endoscope is introduced into the body cavity, in such a condition that an imaging window and an illumination window are covered by a package. Accordingly, the most optimal image-data correction value can be acquired, and further, it becomes possible to easily acquire the most optimal image-data correction value. Moreover, it becomes possible to verify whether the image-data correction value is most optimal, by visualizing a secondly captured image by a display unit.

### BRIEF DESCRIPTION OF DRAWINGS

FIG. 1 is a schematic view of a configuration of a capsule endoscope system according to an embodiment of the present invention;

FIG. 2 is a sectional side view of a capsule endoscope according to the embodiment of the present invention;

FIG. 3 is a block diagram of the capsule endoscope system according to the embodiment of the present invention;

FIG. 4 is a sectional side view of an external package, an inner package, and a chart portion according to the embodiment of the present invention;

FIG. 5 is a front view of the chart portion according to the embodiment of the present invention;

FIG. 6 is a flowchart of a process procedure of the capsule endoscope system according to the embodiment of the present invention; and

FIG. 7 is a schematic view for describing a chart gradation of a gray chart according to the embodiment of the present invention.

### EXPLANATIONS OF LETTERS OR NUMERALS

1 Capsule endoscope system

2 Capsule endoscope

4 External device

4a Receiving device

50 Package

51 External package

70 Internal package

71 Chart portion

### BEST MODE(S) FOR CARRYING OUT THE INVENTION

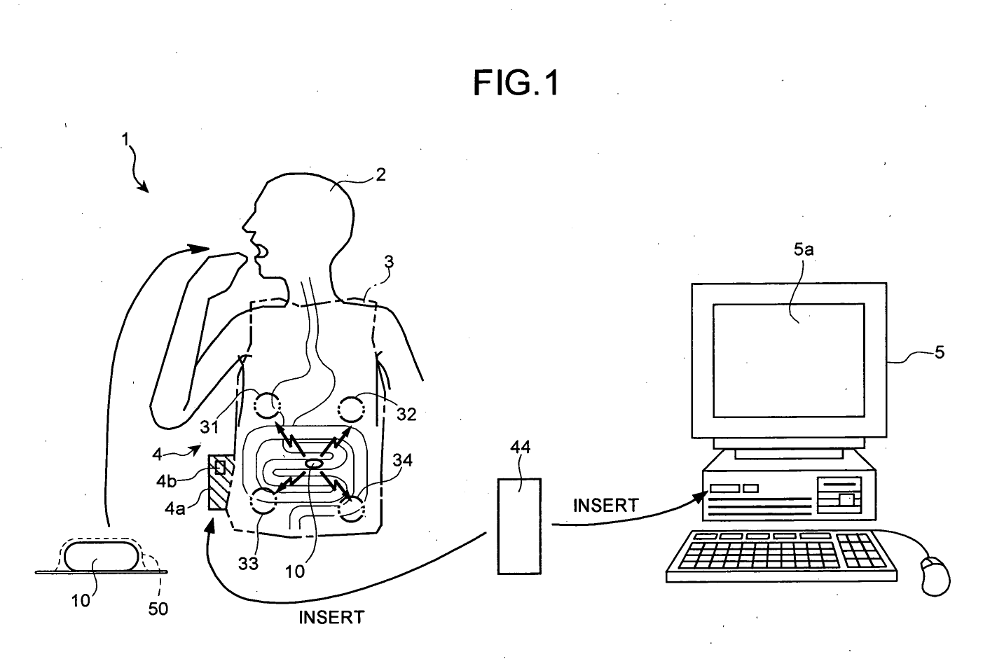

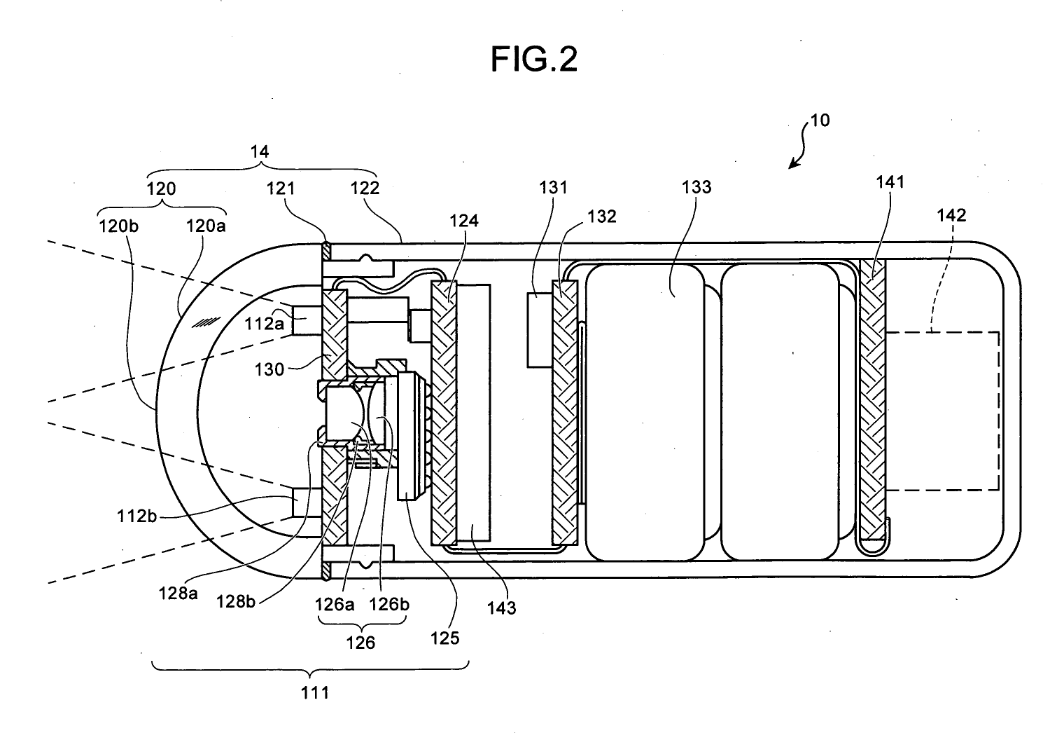

Exemplary embodiments of the present invention will be described below. FIG. 1 is a schematic view of a configuration of a capsule endoscope system according to an embodiment of the present invention. FIG. 2 is a sectional side view of a capsule endoscope according to the embodiment of the present invention.

The capsule endoscope system according to the embodiment of the present invention includes, as shown in FIG. 1, a capsule endoscope 10 that is swallowable from a mouth and captures an image of a body cavity in a process that the capsule endoscope 10 passes through the body cavity such as an esophagus, a stomach, a small intestine, and a colon, and includes an external device 4 that is physically and entirely separated from the capsule endoscope 10 to receive, by a radio communication, image data acquired by the capsule endoscope 10.

The capsule endoscope 10 includes, as shown in FIG. 2, a capsule main body 14 that is a capsule-shaped exterior package, an imaging unit 111 that is arranged inside the capsule main body 14 and captures an image, illuminating units 112a and 112b that illuminate an imaging target region, and a control unit 143 that performs a control of the imaging unit 111 and the illuminating units 112a and 112b or performs a signal process.

For describing each of the units in detail, the capsule main body 14 is in a size swallowable by a human and formed by watertightly bonding a substantially hemisphere-shaped tip portion 120 to a cylindrical-shaped body portion 122. The tip portion 120 and the body portion 122 make the tip portion 120 and the body portion 122 be watertightly bonded via an O-ring 121. It is needles to say that the shape and the configuration of the capsule main body 14 are not limited to those in the present embodiment.

The imaging unit 111 includes a solid-state image sensor 125 arranged on an imaging board 124 and an imaging lens 126 that forms an image of a subject image on the solid-state image sensor 125. A CCD is used as the solid-state image sensor 125. It is obvious that the solid-state image sensor 125 is not limited to the CCD and can be a complementary metal-oxide semiconductor (CMOS). The imaging lens 126 includes a first lens 126a arranged on a fixed frame 128a and positioned on the side of the subject, and a second lens 126b arranged on a movable frame 128b and positioned on the side of the CCD. The imaging lens 126 performs a focus adjustment by shifting the second lens 126b backward and forward. A transparent imaging window 120b is formed on the tip portion 120 of the capsule main body 14.

The illuminating units 112a and 112b are formed by arranging a light emitting diode (LED) as a light emitter on an illuminating board 130. Particularly, according to the present embodiment, four light emitters are arranged in a surrounding area of the imaging lens 126. A transparent illumination window 120a is formed on the tip portion 120 of the capsule endoscope 10 so that a light emitted from the light emitter is output to the outside of the capsule main body 14. Further, according to the present embodiment, the entire tip portion 120 of the capsule endoscope 10 is made of a transparent resign, and the illumination window 120a and the imaging window 120b are partly overlapped. It is obvious that the illumination window 120a and the imaging window 120b can be clearly separated from each other in the configuration.

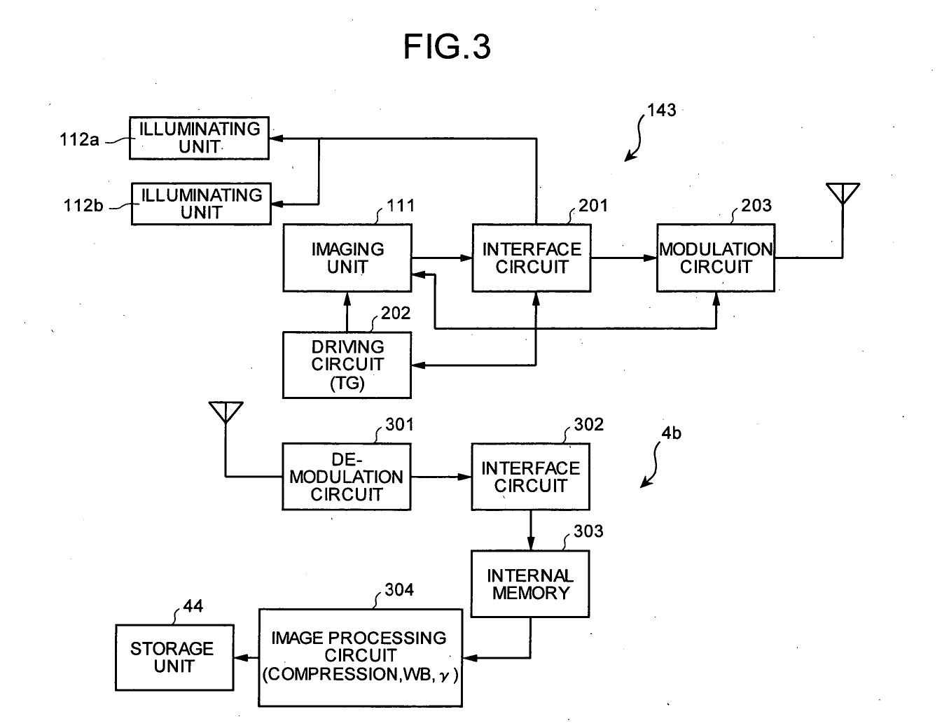

A configuration of the control unit 143 will be explained with reference to FIG. 3. FIG. 3 is a block diagram of the capsule endoscope system according to the present embodiment. Specifically, FIG. 3 describes a flow of image data from the control unit 143 in the capsule endoscope 10 to a receiving-device main body 4b in the external device 4. The control unit 143 includes an interface circuit 201 and a driving circuit 202, performs various signal processes of, for example, generating image data in cooperation with the imaging unit 111, performs a timing-generator function of enabling the imaging unit 111 to capture an image at a predetermined time interval, and stores various parameters related to, for example, a line or a frame. Therefore, according to the present embodiment, two images are captured per second by the imaging unit 111, image data is generated by performing the signal process such as a color correction, and the image data is sequentially transmitted by radio to the external device 4.

A radio transmission is performed, as shown in FIG. 2 and FIG. 3, by a radio transmitting unit configured by providing an antenna on a radio board 141. When the radio transmission is performed, the image data is transmitted and received between the capsule endoscope 10 and the external device 4 by a modulation circuit 203 arranged in the capsule endoscope 10 and a demodulation circuit 301 arranged in the external device 4, using a predetermined communication method (i.e., phase shift keying (PSK), minimum shift keying (MSK), gaussian minimum shift keying (GMSK), quadrature minimum shift keying (QMSK), and amplitude shift keying (ASK)).

On the other hand, as shown in FIG. 1, the external device 4 includes a receiving device 4a that includes antennas 31, 32, 33, and 34 arranged on proper positions of a chest or a abdomen of a subject 2 and the receiving-device main body 4b.

The antennas 31, 32, 33, and 34 are connected to the receiving-device main body 4b with wires. The antennas 31, 32, 33, and 34 are arranged inside a jacket 3 that is wearable on an upper body of the subject 2. Particularly, according to the present embodiment, the antennas 31, 32, 33, and 34 are arranged inside the jacket 3 and an antenna with the highest receiver sensitivity corresponding to a movement of the capsule endoscope inside the body cavity can be selected by a selector arranged in the receiving-device main body 4b to receive the image data. The jacket 3 is an example of the present embodiment, and the present invention is not thus limited.

As shown in FIG. 3, the receiving-device main body 4b includes the interface circuit 302, an internal memory 303, an image processing circuit 304 (the image processing circuit 304 according to the present embodiment includes a function as a control unit explained later, however, it is acceptable to configure the image processing circuit 304 and the control unit independently). The receiving-device main body 4b performs the signal process of, for example, data reproduction or data compression for image data received by a predetermined communication method, and stores the image data in a storage unit 44. As the signal process, a color correction, a y correction, and a color process are performed. The storage unit 44 is configured by connecting a removable storage medium such as a compact flash (CF) memory card or a memory stick to the receiving-device main body 4b. As shown in FIG. 1, by causing an information processing device 5 such as a personal computer to read the storage medium, a body cavity image visualized from the image data stored in the storage medium can be displayed on a display unit 5a such as a monitor.

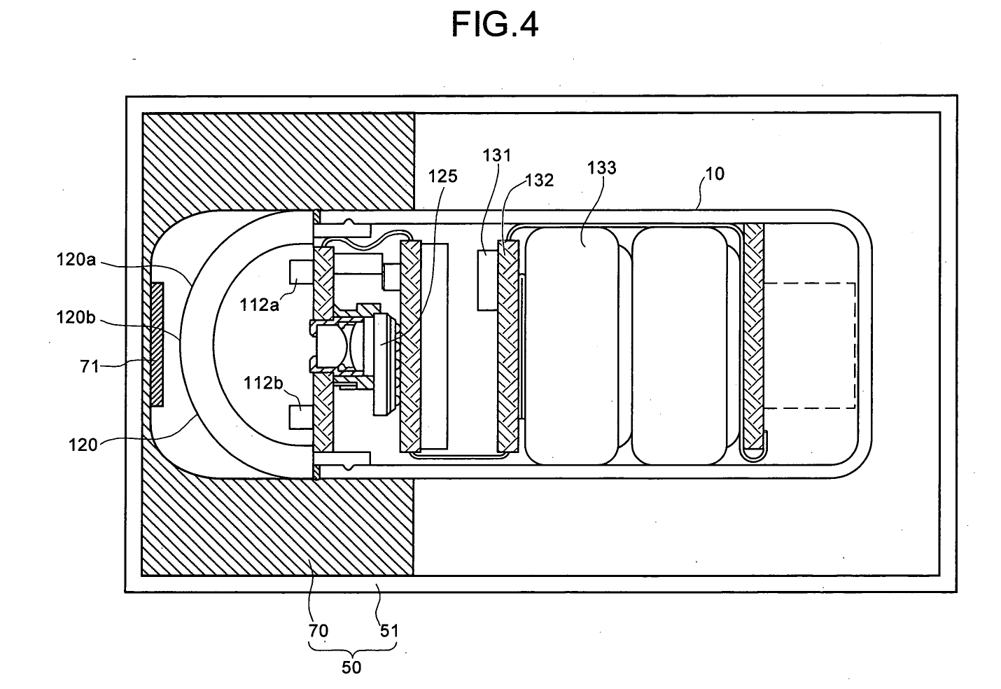

Therefore, the capsule endoscope system 1 according to the present embodiment includes, as shown in FIG. 4, a package 50 that covers at least one of the imaging window 120b and the illumination window 120a of the capsule endoscope 10, a power-supply initiating unit that causes the capsule endoscope 10 to initiate power supply in such a state that the imaging window 120b and the illumination window 120a are covered by the package 50, and a chart portion 71 arranged inside the package 50 so that the chart portion 71 faces the imaging window 120b.

Each of the units will be described below in detail. The package 50 includes an external package 51 in which the capsule endoscope 10 is installed and an internal package 70 that is arranged inside the external package 51 and covers the capsule endoscope 10.

The external package 51 enables to store the capsule endoscope 10 in such a state that the capsule endoscope 10 is shielded from the outside before the capsule endoscope 10 is introduced inside the body of the subject 2. Although the external package 51 having a rectangular-shaped cross section is used as shown in the drawings, the present invention is not thus limited.

On the other hand, the internal package 70 is made of a soft material having a light blocking effect. Further, the internal package 70 completely covers the illumination window 120a and the imaging window 120b of the capsule endoscope 10 to prevent a light from the outside from irradiating the illumination window 120a and the imaging window 120b, when the capsule endoscope 10 is removed from the external package 51. The internal package 70 is removably set on the tip portion 120 of the capsule main body 14 so that a relative position between the chart portion 71 and the capsule main body 14 is kept constant.

The power-supply initiating unit is for initiating to start supplying power to, for example, the imaging unit 111 and the illuminating unit 112, specifically in such a state that the external package 51 is removed and the internal package 70 covers the illumination window 120a and the imaging window 120b of the capsule endoscope 10. The power-supply initiating unit includes an ON/OFF switch 131, an external magnet, and an internal magnet.

The ON/OFF switch 131 is arranged on a power board unit 132 of the capsule endoscope 10 for causing the battery 133 (i.e., silver oxide battery), which is also arranged on the power board unit 132, to start supplying power to the capsule endoscope 10.

The external magnet is arranged in the package 50 in which the capsule endoscope 10 is installed to energize the ON/OFF switch 131 to an OFF state.

On the other hand, the internal magnet is arranged in a neighboring area of the ON/OFF switch 131 inside the capsule main body 14 to energize the ON/OFF switch 131 to an ON state. Therefore, it is configured so that when the capsule endoscope 10 is separated from the external magnet by, for example, removing the capsule endoscope 10 from the package 50, the ON/OFF switch 131 in the OFF state is switched to the ON state. The switch mechanism is not limited to the above described ones. As for the battery 133, a rechargeable battery or an external-power supply type accumulator can be acceptable.

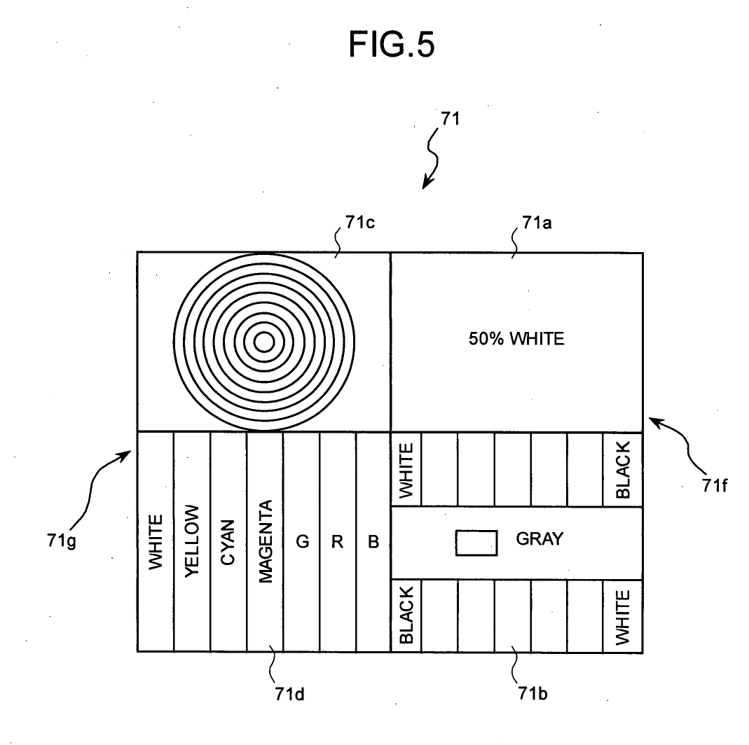

The chart portion 71 includes a chart portion 71f for acquiring an image-data correction value and a chart portion 71g for a verification, and are arranged inside the internal package 70. The chart portion 71 is in a substantially rectangular-plate shape and includes the chart portion 71f for acquiring the image-data correction value and the chart portion 71g for the verification, on a surface at a side on which the imaging window 120b is arranged. Specifically, according to the present embodiment, the chart portion 71f for acquiring the image-data correction value includes a chart for acquiring a color-correction value and a chart for acquiring a gradation-correction value, while the chart portion 71g for the verification includes a resolution chart and a color bar. As shown in FIG. 5, the chart portion 71 is divided at each of middle positions on a vertical and a horizontal side, resulting in being divided into four areas 71a to 71d. The first area 71a is the chart for acquiring the color-correction value, specifically, according to the present embodiment, a white chart (50% white) for acquiring a white-balance correction value. The second area 71b is the chart for acquiring the gradation-correction value, specifically, according to the present embodiment, a gray scale chart. The gray scale chart has an upper column, a middle column, and a lower column. In the upper column, colors corresponding to each of brightness (gradation) from white to black are displayed at, for example, 10 levels from the left side to the right side of the chart. In the middle column, solid gray is displayed. In the lower column, colors corresponding to each of brightness (gradation) from black to white are displayed at, for example, 10 levels from the left side to the right side of the chart. The third area 71c is the resolution chart as the chart for the verification. More specifically, the third area 71c is a so-called circular zone chart in which a plurality of concentric circles is formed with an arrangement in which the concentric circles become denser toward an outer circumference. The fourth area 71d is the color bar as the chart for the verification, in which white, yellow, cyan, magenta, green (G), red (R), and blue (B) are displayed in that order from the left side of the color bar. A layout of arranging each of the white chart, the gray scale chart, the resolution chart, and the color bar is not limited to one described in the present embodiment and various modification can be acceptable, for example, the color bar can be arranged in the center.

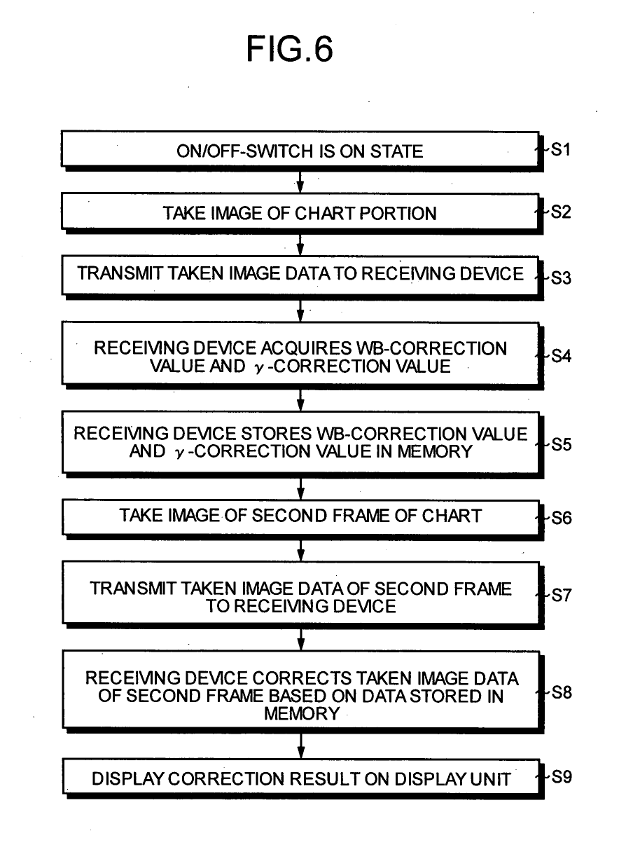

An operation of the capsule endoscope 10 according to the present embodiment is explained with reference to FIG. 6. The capsule endoscope 10 is removed from the external package 51. At the same time, the ON/OFF switch is switched to the ON state (step S1).

Accordingly, a power is supplied from the battery 133 to the radio unit including the imaging unit 111, the illuminating units 112a and 112b, an antenna 142, and the radio board 141, and a light emitter emits a light. On the other hand, because the imaging unit 111 is configured, as described above, to capture an image of the chart portion 71 illuminated by the illuminating units 112a and 112b at a predetermined time interval, the imaging unit 111 performs a first imaging of the chart portion 71 fixed to the internal package 70 when the predetermined time passed (step S2).

The image data is transmitted to the receiving device 4a via the radio unit including the radio board 141 and the antenna 142 (step S3).

When the receiving device 4a receives the image data of the chart portion 71, the image processing circuit 304 that performs a function as the control unit acquires a white balance (WB)-correction value used for a white-balance correction from the white chart, based on the image data, and acquires a y-correction value used for a gradation (Y) correction from the gray scale chart (step S4). Thereafter, the image processing circuit 304 stores the WB-correction value and the y-correction value in the internal memory 303 of the receiving-device main body 4b (step S5).

At this state, values α and β as the WB-correction values are obtained as the following. Namely, each of signal levels (brightness) corresponding to R, G, and B in a predetermined area (i.e., an area of 16×16) of the chart portion used for acquiring the WB-correction value is extracted, and α and β are obtained so that α and β satisfy each of equations,
(average of signal level of G)=α(average of signal level of R), and
(average of signal level of G)=β(average of signal level of B).

Further, the γ-correction value is determined by the following Equation.
Y=X^{γ} [Equation 1]

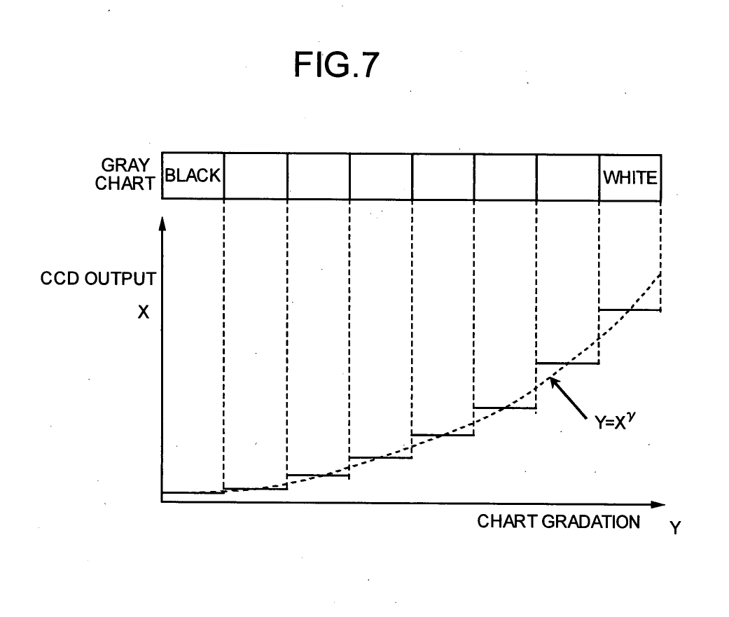

Namely, γ is determined by performing, for example, a fitting from values of X and Y so that the above equation becomes true. At this state, as shown in FIG. 7, Y is the chart gradation determined by the gray chart, while X is a CCD output.

The values α, β, and γ can be obtained by other ways.

Thereafter, the capsule endoscope 10 performs a second imaging of the chart portion 71 when the predetermined time passed (step S6), and transmits the image data to the receiving device 4a (step S7).

When the receiving device 4a receives the image data of the second imaging, the image processing circuit 304 that performs a function as the control unit performs the WB-correction and the γ-correction to the image data based on the correction value α, β, and γ (step S8), and stores the processed image data in the storage unit 44.

The image data stored in the storage unit 44 at step S8 is visualized and displayed by the display unit 5a, by causing the information processing device 5 to read the image data (step S9). Accordingly, the image displayed on the display unit 5a can be viewed and it becomes possible to confirm that the correction has been properly performed, by checking an image corresponding to the fourth area (color bar) 71d or an image corresponding to the third area (resolution chart) 71c included in the displayed image.
(Note 4) The capsule endoscope according to claim 2 or claim 3, wherein the chart portion for acquiring the image-correction value includes a chart for acquiring a color-correction value and the chart is a white chart.
Effect: a color-correction value with a good color-reproduction effect can be obtained.
(Note 5) The capsule endoscope according to any one of claim 2, claim 3, and note 4, wherein the chart portion for acquiring the image-correction value includes a chart for acquiring a gradation-correction value and the chart is a gray scale chart.
(Note 6) The capsule endoscope according to any one of claim 2, claim 3, note 4, and note 5, further comprising a chart portion for a verification, arranged inside the internal package so that the chart portion faces the imaging window.
(Note 7) The capsule endoscope according to any one of claim 2, claim 3, notes 4 to 6, wherein the chart for the verification includes a color bar portion and a resolution chart portion.
(Note 8) The capsule endoscope according to note 7, wherein the chart for the verification and the chart for acquiring the correction value are arranged on a same member.

### INDUSTRIAL APPLICABILITY

As described, the capsule endoscope system and the capsule endoscope according to an embodiment of the present invention are suitable for, i.e., a swallowable capsule endoscope used in a medical field.

1. A capsule endoscope system comprising:
a capsule endoscope that contains an imaging unit, an illuminating unit capable of illuminating an imaging target region, and a transmitting unit capable of transmitting image data obtained by the imaging unit to an outside;
an external device separated from the capsule endoscope, the external device including a receiving unit that receives the image data and a display unit capable of visualizing and displaying the image captured by the imaging unit;
a package that covers at least one of an imaging window and an illumination window of the capsule endoscope, in such a state that an external light can be shielded;
a power-supply initiating unit capable of initiating a power supply to the imaging unit and the illuminating unit, in such a state that the imaging window and the illumination window are covered by the package; and
a chart portion for acquiring an image-data correction value, arranged inside the package so that the chart portion faces the imaging window, wherein the image-data correction value is acquired from an image data obtained by performing a first imaging of the chart portion, in such a state that the imaging window and the illumination window are covered by the package,
an image data obtained by performing a second imaging after the first imaging is corrected by the obtained image-data correction value, in such a state that the imaging window and the illumination window are covered by the package, and
the image data is visualized and displayed on the display unit.

2. A capsule endoscope comprising:
a capsule main body that accommodates therein an imaging unit that is automatically driven to start by supplying power, and an illuminating unit, capable of illuminating an imaging target region, that is automatically driven to start by supplying power;
a package that covers at least one of an imaging window and an illumination window of the capsule main body;
a power-supply initiating unit capable of initiating a power supply to the illuminating unit and the imaging unit, in such a state that the illumination window and the imaging window are covered by the package; and
a chart portion for acquiring an image-data correction value, arranged inside the package so that the chart portion faces the imaging window.

3. The capsule endoscope according to claim 2, wherein
the package includes an external package that accommodates therein the capsule main body, and an internal package that is arranged inside the external package and covers at least one of the imaging window and the illumination window of the capsule main body,
the chart portion for acquiring the image-data correction value is arranged inside the internal package, and
the power-supply initiating unit initiates power supply when the external package is removed.

4. The capsule endoscope according to claim 2, wherein the chart portion for acquiring the image-correction value includes a chart for acquiring a color-correction value and the chart is a white chart.

5. The capsule endoscope according to claim 2, wherein the chart portion for acquiring the image-correction value includes a chart for acquiring a gradation-correction value and the chart is a gray scale chart.

6. The capsule endoscope according to claim 2, further comprising a chart portion for a verification, arranged inside the internal package so that the chart portion faces the imaging window.

7. The capsule endoscope according to claim 6, wherein the chart for the verification includes a color bar portion and a resolution chart portion.

8. The capsule endoscope according to claim 7, wherein the chart for the verification and the chart for acquiring the correction value are arranged on a same member.
